(19) United States
(12) Patent Application Publication
(10) Pub. No.: US 2025/0222172 A1
(43) Pub. Date: Jul. 10, 2025

(54) SKIN GRAFT COMPOSITION AND METHOD OF MAKING AND USING SAME

(71) Applicant: BIOLAB HOLDINGS, INC., Mesa, AZ (US)

(72) Inventors: Carlos ENCINAS, Scottsdale, AZ (US); Laura Andrea GÓMEZ LEÓN, Bogotá (CO); Luminita SKIBA, Scottsdale, AZ (US); Rodrigo Foción SOTO PAREJA, Bogotá (CO)

(73) Assignee: BIOLAB HOLDINGS, INC., Mesa, AZ (US)

(21) Appl. No.: 18/852,984

(22) PCT Filed: Mar. 30, 2023
(86) PCT No.: PCT/US2023/016981

§ 371 Date: Sep. 30, 2024

Related U.S. Application Data
(60) Provisional application No. 63/326,261, filed on Mar. 31, 2022.

(51) Int. Cl. A61L 27/36; A61L 27/38; A61L 27/44; A61L 27/60

(52) U.S. Cl. CPC A61L 27/362; A61L 27/3687; A61L 27/3691; A61L 27/3804; A61L 27/3813; A61L 27/446; A61L 27/60; A61L 2430/34

(57) ABSTRACT

The invention provides a method for preparing a skin graft composition comprising (a) obtaining, from a subject requiring a skin layer patch or dressing, a skin sample, (b) placing the skin sample in a container with a sterile medium, (c) separating fibroblasts and keratinocytes from the skin sample to obtain a solution comprising fibroblasts and keratinocytes or reshaping and resizing the skin sample to obtain a solution comprising the reshaped and resized skin sample, and (d) combining the fibroblasts and the keratinocytes with a matrix to thereby form a skin graft composition. The invention also provides a method for providing a skin graft composition to a subject, as well as a method of providing a skin layer patch or dressing, or a skin graft, to a subject in need thereof.

### BACKGROUND OF THE INVENTION

Cutaneous tissue loss due to burns, injuries, disease, infection, or other traumas, often requires treatment by coating the affected area. For example, physicians use skin grafts obtained from the patient's own skin or from an external source. Skin grafts obtained from the patient's own skin, however, typically generate a large scar in the area from which the skin is taken, e.g., the thighs, back, or buttocks. Skin grafts also can be taken from donors or from dead bodies, which also pose issues, such as numerous screenings to determine the health of the donor, the absence of communicable diseases, and the compatibility of the donor skin graft to the patient skin, as well as potential immunological rejection of the donor skin graft by the patient.

Current skin grafts also require surgery with general or partial anesthesia which can generate potential complications such as severe pain, infection, bleeding, additional surgeries, and, although very rare, death. Such surgeries also require additional hardships, such as hospitalization, lengthy healing, and treatment of scars in the donor area.

Thus, new skin graft compositions are still needed.

### BRIEF SUMMARY OF THE INVENTION

The invention provides a method for preparing a skin graft composition comprising (a) obtaining, from a subject requiring a skin layer patch or dressing, a skin sample, (b) placing the skin sample in a container with a sterile medium, (c) separating fibroblasts and keratinocytes from the skin sample to obtain a solution comprising fibroblasts and keratinocytes or reshaping and resizing the skin sample to obtain a solution comprising the reshaped and resized skin sample, and (d) combining the solution comprising the fibroblasts and the keratinocytes or the solution comprising the reshaped and resized skin sample with a matrix to thereby form a skin graft composition. The invention also provides a method for providing a skin graft composition to a subject, as well as a method of providing a skin layer patch or dressing, or a skin graft, to a subject in need thereof.

### DETAILED DESCRIPTION OF THE INVENTION

The inventive skin graft composition is prepared by a method comprising, consisting essentially of, or consisting of (a) obtaining, from a subject requiring a skin layer patch or dressing, a skin sample, (b) placing the skin sample in a container with a sterile medium, (c) separating fibroblasts and keratinocytes from the skin sample to obtain a solution comprising fibroblasts and keratinocytes or reshaping and resizing the skin sample to obtain a solution comprising the reshaped and resized skin sample, and (d) combining the solution comprising the fibroblasts and the keratinocytes or the solution comprising the reshaped and resized skin sample with a matrix to thereby form a skin graft composition.

The method comprises obtaining, from a subject requiring a skin layer patch or dressing, a skin sample. The subject can be any mammal, preferably a human, that requires a skin layer patch or dressing. The skin layer patch or dressing can be a skin graft. The skin sample can be any suitable sample. The skin sample can be obtained from any suitable place on a patient. In some embodiments, the skin sample is obtained from the head, back, buttocks, or legs, e.g., the thighs. In some embodiments, the skin sample comprises the epidermis, dermis, and subcutaneous tissue. In other embodiments, the skin sample comprises the epidermis and dermis. In other embodiments, the skin sample comprises the epidermis, and at least a part of the dermis, but does not comprise the entire dermis.

The skin sample can be obtained in any suitable manner using any suitable tool. For example, in some embodiments, the skin sample is obtained using a razor or similar tool, a skin punch (also called a biopsy punch or surgical punch), or a scalpel. In other embodiments, the skin sample is obtained using a dermatome such as a Goulian knife or an air dermatome.

The skin sample can be of any suitable size. In some embodiments, the skin sample has a surface area of at least 1 mm². For example, in some embodiments, the skin sample has a surface area of at least 1 mm², at least 2 mm², at least 3 mm², at least 4 mm², at least 5 mm², at least 6 mm², at least 7 mm², at least 8 mm², at least 9 mm², at least 10 mm², at least 11 mm², at least 12 mm², at least 13 mm², at least 14 mm², at least 15 mm², at least 16 mm², at least 17 mm², at least 18 mm², at least 19 mm², at least 20 mm², at least 100 mm², at least 500 mm², at least 1000 mm², at least 1500 mm², at least 2000 mm², at least 2500 mm², at least 3000 mm², at least 4000 mm², at least 5000 mm², or at least 10000 mm², or a surface area between any two of the aforementioned maximum values. In some embodiments, the sample has a length of between 1 and 10 mm, e.g., 1 mm, 2 mm, 3 mm, 4 mm, 5 mm, 6 mm, 7 mm, 8 mm, 9 mm, or 10 mm, or a length within a range defined by any two of the foregoing values. In some embodiments, the sample has a width between 1 and 10 mm, e.g., 1 mm, 2 mm, 3 mm, 4 mm, 5 mm, 6 mm, 7 mm, 8 mm, 9 mm, 10 mm, or a width within a range defined by any two of the foregoing values. In some embodiments, the skin sample has a thickness of 2-3 mm. For example, in some embodiments the sample has a thickness of 2 mm, 2.1 mm, 2.2 mm, 2.3 mm, 2.4 mm, 2.5 mm, 2.6 mm, 2.7 mm, 2.8 mm, 2.9 mm, or 3 mm, or a thickness within a range defined by any two of the foregoing values. In some embodiments, the skin sample has a thickness equal to or less than 1 mm. For example, in some embodiments, the sample has a thickness of 1 mm, 0.9 mm, 0.8 mm, 0.7 mm, 0.6 mm, 0.5 mm, 0.4 mm, 0.3 mm, 0.2 mm, or 0.1 mm, or a thickness within a range defined by any two of the foregoing values.

In some embodiments, the method comprises dissecting the skin sample prior to placing the skin sample in a container with a sterile medium. The skin sample can be dissected in any suitable manner. In some embodiments, the skim sample is dissected using a scalpel or knife.

The method comprises placing the skin sample into a container with a sterile medium. The container can be any suitable container. In some embodiments, the container is a glass or plastic container. In some embodiments, the container is a cryovial or test tube. In other embodiments, the container is a cell culture flask or roller-bottle. In some embodiments, the container is a petri dish or a cell culture plate. In other embodiments, the container is a cell culture insert placed in a cell culture plate.

The sterile medium can be any suitable sterile medium. A sterile medium is a medium that does not contain any viable bacteria or other cells that would grow and increase in number through cell division. In some embodiments, the sterile medium is a saline solution. In some embodiments, the saline solution is phosphate buffered saline (PBS). In other embodiments, the sterile medium is Dulbecco's Modified Eagle Medium (DMEM). In other embodiments, the sterile medium is Ham's F-12 (F12), Ham's F-10 (F10), Iscove's Modification of DMEM (IDMEM), MEDIUM 199, Alpha MEM, Basal Medium Eagle with Earle's BSS: Cyroprotective Medium (Freezing Medium), DMEM:F12 1:1, with L-Glutamine, Glasgow's MEM with L-glutamine (GMEM), IMDM with HEPES, with or without L-Glutamine: L-15 (Leibovitz), without L-Glutamine, McCoy's 5A Modified Medium, Medium 199, MEM Eagle, MEM Eagle-Earle's BSS, with or without L-Glutamine, MEM Eagle-Hanks BSS, NCTC-109, Richter's CM Medium, RPMI 1640 with HEPES, RPMI 1640. In some embodiments, the sterile medium is a combination of sterile mediums, such as a combination of one or more of the aforementioned sterile mediums. The amount of sterile medium can be any suitable amount of sterile medium. In some embodiments, the amount of sterile medium is 1-10000 μl per mm² skin sample, e.g., 1 μl per mm² skin sample, 500 μl per mm² skin sample, 1000 μl per mm² skin sample, 1500 μl per mm² skin sample, 2000 μl per mm² skin sample, 2500 μl per mm² skin sample, 3000 μl per mm² skin sample, 3500 μl per mm² skin sample, 4000 μl per mm² skin sample, 4500 μl per mm² skin sample, 5000 μl per mm² skin sample, 5500 μl per mm² skin sample, 6000 μl per mm² skin sample, 6500 μl per mm² skin sample, 7000 μl per mm² skin sample, 7500 μl per mm² skin sample, 8000 μl per mm² skin sample, 8500 μl per mm² skin sample, 9000 μl per mm² skin sample, 9500 μl per mm² skin sample, or 10000 μl per mm² skin sample, or an amount of sterile medium that is within a range defined by any two of the foregoing values.

The skin sample can be maintained in the container with the sterile medium for any suitable period of time prior to separating the fibroblasts and the keratinocytes from the skin sample or reshaping and resizing the skin sample. In some embodiments, the skin sample is maintained in the container for up to 72 hours. For example, in some embodiments, the skin sample is maintained in the container for 0 hours, 8 hours, 12 hours, 16 hours, 20 hours, 24 hours, 28 hours, 32 hours, 36 hours, 40 hours, 44 hours, 48 hours, 52 hours, 56 hours, 60 hours, 64 hours, 68 hours, or 72 hours, or for a time period within a range defined by any two of the foregoing values. The skin sample can be maintained in the container with the sterile medium at any suitable temperature. In some embodiments, the skin sample is maintained in the container at a temperature of 5-40° C., e.g., 5-37° C. In some embodiments, the skin sample is maintained in the container at a temperature of 20-40° C. For example, in some embodiments, the skin sample is maintained in the container at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or at a temperature within a range defined by any two of the foregoing values. In a preferred embodiment, the skin sample is maintained in the container at a temperature of 37° C.

In some embodiments, the method further comprises adding additional sterile medium to the container prior to separating the fibroblasts and the keratinocytes from the skin sample or reshaping and resizing the skin sample. The additional sterile medium can be any suitable sterile medium. The additional sterile medium can be the same as or different from the first amount of sterile medium added to the container. A sterile medium is a medium that does not contain any viable bacteria or other cells that would grow and increase in number through cell division. In some embodiments, the sterile medium is a saline solution. In some embodiments, the saline solution is phosphate buffered saline (PBS). In other embodiments, the sterile medium is Dulbecco's Modified Eagle Medium (DMEM). In other embodiments, the sterile medium is Ham's F-12 (F12), Ham's F-10 (F10), Iscove's Modification of DMEM (IDMEM), MEDIUM 199, Alpha MEM, Basal Medium Eagle with Earle's BSS: Cyroprotective Medium (Freezing Medium), DMEM:F12 1:1, with L-Glutamine, Glasgow's MEM with L-glutamine (GMEM), IMDM with HEPES, with or without L-Glutamine: L-15 (Leibovitz), without L-Glutamine, McCoy's 5A Modified Medium, Medium 199, MEM Eagle, MEM Eagle-Earle's BSS, with or without L-Glutamine, MEM Eagle-Hanks BSS, NCTC-109, Richter's CM Medium, RPMI 1640 with HEPES, RPMI 1640. In some embodiments, the sterile medium is a combination of sterile mediums, such as a combination of one or more of the aforementioned sterile mediums. The amount of additional sterile medium can be any suitable amount. In some embodiments, the amount of additional sterile medium is 1-10000 μl per mm² skin sample, e.g., 1 μl per mm² skin sample, 500 μl per mm² skin sample, 1000 μl per mm² skin sample, 1500 μl per mm² skin sample, 2000 μl per mm² skin sample, 2500 μl per mm² skin sample, 3000 μl per mm² skin sample, 3500 μl per mm² skin sample, 4000 μl per mm² skin sample, 4500 μl per mm² skin sample, 5000 per mm² skin sample, 5500 μl per mm² skin sample, 6000 μl per mm² skin sample, 6500 μl per mm² skin sample, 7000 μl per mm² skin sample, 7500 μl per mm² skin sample, 8000 μl per mm² skin sample, 8500 μl per mm² skin sample, 9000 μl per mm² skin sample, 9500 μl per mm² skin sample, or 10000 μl per mm² skin sample, or an amount of sterile medium that is within a range defined by any two of the foregoing values.

The method comprises separating the fibroblasts and keratinocytes from the skin sample to obtain a solution comprising fibroblasts and keratinocytes or reshaping and resizing the skin sample to obtain a solution comprising the reshaped and resized skin sample. In some embodiments, the method comprises separating the fibroblasts and keratinocytes from the skin sample to obtain a solution comprising fibroblasts and keratinocytes. The fibroblasts and keratinocytes can be separated from the skin sample by any suitable method. In some embodiments, the fibroblasts and keratinocytes are separated from the skin sample by mechanical, chemical, or enzymatic extraction. In some embodiments, the fibroblasts and keratinocytes are separated from the skin sample by mechanical extraction. In some embodiments, the fibroblasts and keratinocytes are separated from the skin by enzymatic extraction. In some embodiments, only one type of extraction is used. For example, in some embodiments, mechanical, chemical, or enzymatic extraction is used. In other embodiments, two or more types of extraction are used. For example, in some embodiments, mechanical and chemical extractions, mechanical and enzymatic extractions, or chemical and enzymatic extractions are used. In other embodiments, three or more types of extraction are used. For example, in some embodiments, mechanical, chemical, and enzymatic extractions are used.

In some embodiments, mechanical extraction is used to separate or dissociate the fibroblasts and keratinocytes from the skin sample. Any suitable method of mechanical extraction can be used. For example, in some embodiments, mechanical extraction is shaking, stirring, mixing, grinding, or mechanical pipetting.

In some embodiments, the container comprising the skin sample and sterile medium is shaken. The container comprising the skin sample and the sterile medium is shaken for any suitable period of time to separate the fibroblasts and keratinocytes from the remainder of the skin sample. In some embodiments, the container comprising the skin sample and the sterile medium is shaken for up to 1800 seconds (s). In some embodiments, the container comprising the skin sample and the sterile medium is shaken for up to one of the lengths of time of 100 s, 200 s, 300 s, 400 s, 500 s, 600 s, 700 s, 800 s, 900 s, 1000 s, 1100 s, 1200 s, 1300 s, 1400 s, 1500 s, 1600 s, 1700 s, or 1800 s, or for a time period defined by any two of the foregoing values. For example, in some embodiments, the container comprising the skin sample and the sterile medium is shaken for 5-100 s. In some embodiments, the container comprising the skin sample and the sterile medium is shaken for up to one of the lengths of time of 10 s, 20 s, 30 s, 40 s, 50 s, 60 s, 70 s, or 80 s, or for a time period defined by any two of the foregoing values. For example, in some embodiments, the container comprising the skin sample and the sterile medium is shaken for 30-40 s, e.g., 37 s. In other embodiments, the container comprising the skin sample and the sterile medium is shaken for 60-70 s, e.g., 64 s. The container comprising the skin sample and the sterile medium is shaken in any suitable manner by any suitable device. In some embodiments, the container is shaken manually. In some embodiments, the container is shaken using a device, such as a laboratory shaker or dissociator.

In other embodiments, the skin sample and sterile medium in the container is stirred or mixed. The skin sample and the sterile medium in the container is stirred or mixed for any suitable period of time to separate the fibroblasts and keratinocytes from the remainder of the skin sample. In some embodiments, the skin sample and the sterile medium in the container is stirred or mixed for up to 1800 seconds (s). In some embodiments, the skin sample and the sterile medium in the container is stirred or mixed for up to one of the lengths of time of 100 s, 200 s, 300 s, 400 s, 500 s, 600 s, 700 s, 800 s, 900 s, 1000 s, 1100 s, 1200 s, 1300 s, 1400 s, 1500 s, 1600 s, 1700 s, or 1800 s, or for a time period defined by any two of the foregoing values. For example, in some embodiments, the skin sample and the sterile medium in the container is stirred or mixed for 5-100 s. In some embodiments, the skin sample and the sterile medium in the container is stirred or mixed for up to one of the lengths of time of 10 s, 20 s, 30 s, 40 s, 50 s, 60 s, 70 s, or 80 s, or for a time period defined by any two of the foregoing values. For example, in some embodiments, the skin sample and the sterile medium in the container is stirred or mixed for 30-40 s, e.g., 37 s. In other embodiments, the skin sample and the sterile medium in the container is stirred or mixed for 60-70 s, e.g., 64 s. The skin sample and the sterile medium in the container is stirred or mixed in any suitable manner by any suitable device. In some embodiments, the skin sample and the sterile medium is stirred or mixed manually. In some embodiments, the skin sample and the sterile medium in the container is stirred or mixed, such as a laboratory stirrer, mixer, or dissociator, or by using a mixing rod.

In other embodiments, mechanical extraction is mechanical pipetting. In other embodiments, mechanical extraction is grinding the sample with any suitable grinder or mill.

In some embodiments, chemical extraction is used to separate or dissociate the fibroblasts and keratinocytes from the skin sample. Any suitable method of chemical extraction can be used. In some embodiments, chemical extraction comprises incubating the skin sample and the sterile medium in the container with a chemical for a suitable period of time at a suitable temperature. In some embodiments, the chemical is ethylenediaminetetraacetic acid (EDTA) and/or egtazic acid (EGTA). In some embodiments, the chemical extraction is conducted for a period of time within the range of 1 minute-24 hours. In some embodiments, the chemical extraction is conducted at a temperature of 5-50° C., such as, for example, 5-37° C. In some embodiments, the chemical extraction is conducted at a temperature of 20-40° C., e.g., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or at a temperature within a range defined by any two of the foregoing values. In a preferred embodiment, the chemical extraction is conducted at a temperature of 37° C.

In some embodiments, enzymatic extraction is used to separate or dissociate the fibroblasts and keratinocytes from the sample. Any suitable method of enzymatic extraction can be used. In some embodiments, the skin sample and the sterile medium in the container are incubated with the enzyme for a suitable period of time at a suitable temperature. In some embodiments, the enzyme is trypsin, chymotrypsin, papain, chymopapain, collagenase, elastase, hyaluronidase, Dnase, pronase, and/or dispase. In some embodiments, the enzymatic extraction is conducted for a period of time within the range of 1 minute-24 hours. In some embodiments, the enzymatic extraction is conducted at a temperature of 5-50° C., such as, for example, 5-37° C. In some embodiments, the enzymatic extraction is conducted at a temperature of 20-40° C., e.g., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or at a temperature within a range defined by any two of the foregoing values. In a preferred embodiment, the enzymatic extraction is conducted at a temperature of 37° C.

The solution comprising fibroblasts and keratinocytes that is formed after separating the fibroblasts and keratinocytes from the skin sample can be free, or at least substantially free, of microbes. In some embodiments, the solution comprising fibroblasts and keratinocytes that is formed after separating the fibroblasts and keratinocytes from the skin sample can be free, or at least substantially free, of bacteria. In some embodiments, the solution comprising fibroblasts and keratinocytes that is formed after separating the fibroblasts and keratinocytes from the skin sample can be free, or at least substantially free, of viruses.

In some embodiments, the solution comprising fibroblasts and keratinocytes does not substantially contain, or does not contain, other skin cells. In some embodiments, the solution formed following the separation of fibroblasts and keratinocytes from the skin sample contains bacteria and optionally other microbes. In such embodiments, the method for preparing a skin graft composition further comprises removing the bacteria and optionally any other microbes from the solution formed following the separation of fibroblasts and keratinocytes from the skin sample, such that the solution is free, or at least substantially free, of bacteria and optionally any other microbes. Any suitable method can be used to remove the bacteria and optionally any other microbes. In some embodiments, the bacteria and optionally any other microbes are removed using any suitable antibacterial or antimicrobial agent. In some embodiments, the antibacterial or antimicrobial agent is penicillin, streptomycin, gentamycin sulfate, tobramycin, and/or amphotericin B. As used herein, substantially free of bacteria or optionally any other microbes refers to fewer than 100 bacteria or any other microbes per ml, e.g., fewer than one of the amounts of microbes of 100, 90, 80, 70, 60, 50, 40, 30, 20, or 10, or a number of bacteria or any other microbes within a range defined by any two of the foregoing values.

In some embodiments, the solution comprising the fibroblasts and keratinocytes formed after separating the fibroblasts and keratinocytes from the skin sample also comprises the remaining parts of the skin sample, such as hair follicles, adipocytes, melanocytes, capillaries and non-fibroblast connective tissue cells.

In some embodiments, the method comprises reshaping and resizing the skin sample to obtain a solution comprising the reshaped and resized skin sample. The skin sample can be reshaped and resized in any suitable manner. In some embodiments, the skin sample is reshaped and resized by cutting the skin sample into smaller pieces. The skin sample can be cut using any suitable tool, such as a scalpel. In other embodiments, the skin sample is reshaped and resized using a dissociator. For example, in some embodiments, the container with the skin sample and sterile medium is placed in a dissociator. In some embodiments, the container with the skin sample and sterile medium is placed in a dissociator for any suitable amount of time. In some embodiments, the skin sample is placed in the dissociator for any period of time between 10 seconds and 2 minutes, e.g., 10 seconds, 20 seconds, 30 seconds, 40 seconds, 50 seconds, 60 seconds, 70 seconds, 80 seconds, 90 seconds, 100 seconds, 110 seconds, or 120 seconds, or for a length of time within a range defined by any two of the foregoing values. In some embodiments, the skin sample is placed in the dissociator for 50-60 seconds, such as, e.g., 50 seconds, 51, seconds, 52 seconds, 53 seconds, 54 seconds, 55 seconds, 56 seconds, 57 seconds, 58 seconds, 59 seconds, or 60 seconds, or for a length of time within a range defined by any two of the foregoing values. In some embodiments, the reshaped and resized skin sample maintains all of the layers of the skin sample.

In some embodiments, the skin sample is removed from the container and the sterile medium before reshaping and resizing of the skin sample, and replaced into the container and the sterile medium after reshaping and resizing of the skin sample. In some embodiments, the skin sample is replaced into the empty container, and additional sterile medium, as described herein, is added to the container to form a solution comprising the reshaped and resized skin sample.

In some embodiments, the method for preparing a skin graft composition further comprises filtering and/or centrifuging the solution comprising the fibroblasts and the keratinocytes or the solution comprising the reshaped and resized skin sample to provide a purified solution of the fibroblasts and the keratinocytes or a purified solution comprising the reshaped and resized skin sample. In some embodiments, a purified solution of the fibroblasts and keratinocytes is a solution comprising fibroblasts and keratinocytes that is free, or at least substantially free, of one or more of the following substances: melanocytes, adipocytes, hair follicles, capillaries, and non-fibroblast connective tissue cells. Desirably, the purified solution of the fibroblasts and keratinocytes is a solution comprising fibroblasts and keratinocytes that is free, or at least substantially free, of melanocytes, adipocytes, hair follicles, capillaries, and non-fibroblast connective tissue cells. In some embodiments, a solution that is substantially free of melanocytes, adipocytes, hair follicles, capillaries, and non-fibroblast connective tissue cells is a solution that has less than 10000 melanocytes, adipocytes, hair follicles, capillaries, and non-fibroblast connective tissue cells. In some embodiments, a solution that is substantially free of melanocytes, adipocytes, hair follicles, capillaries, and non-fibroblast connective tissue cells is a solution that has less than 9000, less than 8000, less than 7000, less than 6000, less than 5000, less than 4000, less than 3000, less than 2000, or less than 1000 melanocytes, adipocytes, hair follicles, capillaries, and non-fibroblast connective tissue cells, or melanocytes, adipocytes, hair follicles, capillaries, and non-fibroblast connective tissue cells within a range defined by any two of the foregoing values.

In some embodiments, the method for preparing a skin graft composition further comprises filtering the solution comprising fibroblasts and keratinocytes to provide a purified solution of the fibroblasts and the keratinocytes or filtering the solution comprising the reshaped and resized skin sample to provide a purified solution of the reshaped and resized skin sample. Any suitable filter may be used to provide a purified solution of the fibroblasts and the keratinocytes. In some embodiments, the filter has a pore size of 1-200 microns. In some embodiments, the filter has a pore size of 10-100 microns, e.g., 10 microns, 20 microns, 30 microns, 40 microns, 50 microns, 60 microns, 70 microns, 80 microns, 90 microns, or 100 microns, or a pore size within a range defined by any two of the foregoing values.

In some embodiments, the method for preparing a skin graft composition further comprises centrifuging the solution comprising fibroblasts and keratinocytes to provide a purified solution of the fibroblasts and the keratinocytes or centrifuging the solution comprising the reshaped and resized skin sample to provide a purified solution comprising the reshaped and resized skin sample. The solution comprising fibroblasts and keratinocytes or the solution comprising the reshaped and resized skin sample can be centrifuged for any suitable amount of time at any suitable amount of force. In some embodiments, the solution comprising fibroblasts and keratinocytes or the solution comprising the reshaped and resized skin sample is centrifuged for 1-60 minutes. In some embodiments, the solution comprising fibroblasts and keratinocytes or the solution comprising the reshaped and resized skin sample is centrifuged for 1 minute, 5 minutes, 10 minutes, 15 minutes, 20 minutes, 25 minutes, 30 minutes, 35 minutes, 40 minutes 45 minutes, 50 minutes, 55 minutes, or 60 minutes, or for a time within a range defined by any two of the foregoing values. In some embodiments, the solution comprising fibroblasts and keratinocytes or the solution comprising the reshaped and resized skin sample is centrifuged at 1-400 g. In some embodiments, the solution comprising fibroblasts and keratinocytes or the solution comprising the reshaped and resized skin sample is centrifuged at 1-300 g. In some embodiments, the solution comprising fibroblasts and keratinocytes or the solution comprising the reshaped and resized skin sample is centrifuged at 100-300 g. In some embodiments, the solution comprising fibroblasts and keratinocytes or the solution comprising the reshaped and resized skin sample is centrifuged at 150-300 g. In some embodiments, the solution comprising the fibroblasts and the keratinocytes or the solution comprising the reshaped and resized skin sample is centrifuged at 180-300 g, e.g., 180 g, 190 g, 200 g, 210 g, 220 g, 230 g, 240 g, 250 g, 260 g, 270 g, 280 g, 290 g, or 300 g, or at a force within a range defined by any two of the foregoing values.

In some embodiments, the method comprises centrifuging the purified solution comprising fibroblasts and keratinocytes or the purified solution comprising the reshaped and resized skin sample at least one additional time, e.g., at least one, at least two, at least three, or at least four additional times. The purified solution comprising fibroblasts and keratinocytes or the purified solution comprising the reshaped and resized skin sample can be centrifuged for any suitable period of time at any suitable force. For example, in some embodiments, the purified solution comprising fibroblasts and keratinocytes or the purified solution comprising the reshaped and resized skin sample is centrifuged at least one additional time for 1-60 minutes. In some embodiments, the purified solution comprising fibroblasts and keratinocytes or the purified solution comprising the reshaped and resized skin sample is centrifuged at least one additional time for 1 minute, 5 minutes, 10 minutes, 15 minutes, 20 minutes, 25 minutes, 30 minutes, 35 minutes, 40 minutes 45 minutes, 50 minutes, 55 minutes, or 60 minutes, or for a period of time within a range defined by any two of the foregoing values. In some embodiments, the purified solution comprising fibroblasts and keratinocytes or the purified solution comprising the reshaped and resized skin sample is centrifuged at least one additional time at 1-400 g. In some embodiments, the solution comprising fibroblasts and keratinocytes or the solution comprising the reshaped and resized skin sample is centrifuged at 1-300 g. In some embodiments, the solution comprising fibroblasts and keratinocytes or the solution comprising the reshaped and resized skin sample is centrifuged at 100-300 g. In some embodiments, the purified solution comprising the fibroblasts and the keratinocytes or the purified solution comprising the reshaped and resized skin sample is centrifuged at least one additional time at 150-300 g. In some embodiments, the purified solution comprising the fibroblasts and the keratinocytes or the purified solution comprising the reshaped and resized skin sample is centrifuged at least one additional time at 180-300 g, e.g., 180 g, 190 g, 200 g, 210 g, 220 g, 230 g, 240 g, 250 g, 260 g, 270 g, 280 g, 290 g, or 300 g, or at a force within a range defined by any two of the foregoing values.

In some embodiments, the method comprises filtering the solution comprising the fibroblasts and keratinocytes or the solution comprising the reshaped and resized skin sample to provide a purified solution comprising the fibroblasts and the keratinocytes or a purified solution comprising the reshaped and resized skin sample followed by centrifuging the purified solution comprising the fibroblasts and keratinocytes or the purified solution comprising the reshaped and resized skin sample. In other embodiments, the method comprises filtering the solution comprising the fibroblasts and keratinocytes or the solution comprising the reshaped and resized skin sample to provide the purified solution comprising the fibroblasts and the keratinocytes or the purified solution comprising the reshaped and resized skin sample followed by centrifuging the purified solution comprising the fibroblasts and keratinocytes or the solution comprising the reshaped and resized skin sample at least two times. In some embodiments, the method comprises removing any visible piece of skin from the purified solution of the fibroblasts and the keratinocytes or the solution comprising the reshaped and resized skin sample after centrifuging the first time and before centrifuging the second time.

In some embodiments, the separation of the fibroblasts and keratinocytes from the skin sample results in the fibroblasts and keratinocytes being viable, live, and/or intact. In some embodiments, the separation of the fibroblasts and keratinocytes from the skin sample results in the fibroblasts and keratinocytes being viable. In some embodiments, the separation of the fibroblasts and keratinocytes results in the fibroblasts and keratinocytes being alive. In some embodiments, the separation of the fibroblasts and keratinocytes results in the fibroblasts and keratinocytes being intact. In some embodiments, use of a skin graft composition or skin graft as described herein, wherein the fibroblasts and keratinocytes are viable, live, and/or intact, results in epithelialization, which otherwise is known as the process by which epithelial cells migrate to the top of an open wound to close it. In some embodiments, use of a skin graft composition or skin graft as described herein, wherein the fibroblasts and keratinocytes are viable, live, and/or intact, results in little to no scar tissue formation.

In other embodiments, the separation of the fibroblasts and keratinocytes from the skin sample results in the lysing, death, and/or breaking apart of all, or at least substantially all, of the cells of the skin sample including the fibroblasts and keratinocytes.

In some embodiments, the cells in the solution comprising the reshaped and resized skin sample are lysed following centrifuging. The cells in the reshaped and resized skin sample are lysed by any suitable means.

In some embodiments, use of a skin graft composition or skin graft as described herein, wherein all or substantially all of the cells in the skin sample have been lysed, results in wound closure and scar tissue formation at the place of application. Without being bound by any particular theory, the lysed cells are believed to release fibroblast recruiting proteins into the solution, such that, when applied to a subject as part of a skin graft composition as described herein, the released fibroblast recruiting proteins help create scar tissue at the site of application. In some embodiments, scar tissue formation occurs at a site with an open wound. In some embodiments, the open wound is a wound is a wound that has been open for at least 1 week. For example, in some embodiments, the wound has been open for at least a length of time of one of the values of 1 week, 2 weeks, 3 weeks, 4 weeks, 5 weeks, 6 weeks, 7 weeks, 8 weeks, 9 weeks, 10 weeks, 11 weeks, 12 weeks, 24 weeks, or 36 weeks, or a length of time within a range defined by any two of the foregoing values. In other embodiments, a the wound is a wound that has been open for at least 3 months, e.g., at least a length of time of one of the values of 3 months, 4 months, 5 months, 6 months, 7 months, 8 months, 9 months, 10 months, 11 months, 12 months, 2 years, 5 years, 10 years, 20 years, 30 years, or 40 years, or a length of time within a range defined by any two of the foregoing values. In some embodiments, the open wound is a chronic wound.

The method comprises combining the solution comprising the fibroblasts and the keratinocytes or the solution comprising the reshaped and resized skin sample with a matrix to form a skin graft composition. In some embodiments, the solution comprising the fibroblasts and the keratinocytes is a purified solution comprising the fibroblasts and the keratinocytes, as described herein, which is combined with the matrix to form a skin graft composition. In some embodiments, the solution comprising the reshaped and resized skin sample is a purified solution comprising the reshaped and resized skin sample, as described herein, which is combined with the matrix to form a skin graft composition. In other embodiments, the method further comprises isolating or substantially purifying the fibroblasts and the keratinocytes from the solution comprising the fibroblasts and the keratinocytes and then combining the isolated or substantially purified fibroblasts and keratinocytes with a matrix. In other embodiments, the method further comprises isolating or substantially purifying the reshaped and resized skin sample from the solution comprising the reshaped and resized skin sample and then combining the isolated or substantially purified skin sample with a matrix.

The matrix can be any suitable matrix. In some embodiments, the matrix is a fluid or gel, such as a fluid matrix or gel matrix that conforms to the surface to which the skin graft composition is applied. For example, in some embodiments, the matrix conforms to a wound. In some embodiments, the matrix is a gel matrix. In some embodiments, the matrix is a colloidal solution. In some embodiments, the matrix comprises, consists essentially of, or consists of collagen, gelatin, agarose, cellulose, elastin, fibrin, proteoglycans such as decorin or biglycan, glycoproteins such as tenascin, vitronectin, fibronectin, laminin, or thrombospondin I, glycosaminoglycans (GAG), such as hyaluronic acid (HA), a synthetic polymeric fiber made of a poly-acid such as polyactic, polyglycolic or polyamino acid, polycaprolactone, polyamino acid, polypeptide gel, copolymers thereof, each alone or in combination. In some embodiments, the matrix is a collagen solution. In some embodiments, the collagen solution comprises type I collagen, type II collagen, type III collagen, or type IV collagen. In some embodiments, the collagen solution comprises type I collagen. In some embodiments, the matrix is a fibrin solution. In some embodiments, the matrix is a keratin solution. In some embodiments, the matrix is a solution comprising, consisting essentially of, or consisting of collagen, fibrin, and keratin. In some embodiments, the matrix is a solution comprising, consisting essentially of, or consisting of collagen and fibrin. In some embodiments, the matrix is a solution comprising, consisting essentially of, or consisting of collagen and keratin. In some embodiments, the matrix is a solution comprising, consisting essentially of, or consisting of fibrin and keratin. In some embodiments, the matrix does not comprise any components other than the recited components.

The matrix can have any suitable pH. In some embodiments, the matrix has a pH of 6-8. In some embodiments, the matrix has a pH of 6.8-7.4, e.g., pH of 6.8, 6.9, 7.0, 7.1, 7.2, 7.3, or 7.4, or a pH within a range defined by any two of the foregoing values. The matrix can have any suitable viscosity. In some embodiments, the matrix has a viscosity of 0.5-1000000 cP. In some embodiments, the matrix has a viscosity of 2000-250000 cP. In some embodiments, the matrix has a viscosity of 10000-100000 cP, e.g., 10000 cP, 15000 cP, 20000 cP, 25000 cP, 30000 cP, 35000 cP, 40000 cP, 45000 cP, 50000 cP, 55000 cP, 60000 cP, 65000 cP, 70000 cP, 7500 cP 0, 80000 cP, 85000 cP, 90000 cP, 95000 cP, or 100000 cP, or a viscosity within a range defined by any two of the foregoing values.

After combining the solution or purified solution comprising the fibroblasts and the keratinocytes or the solution or purified solution comprising the reshaped and resized skin sample with the matrix, in some embodiments, the concentration of the matrix in the matrix containing the fibroblasts and keratinocytes or the matrix containing the skin sample is no greater than 100 mg/ml. In some embodiments, the concentration of the matrix is no greater than one of the values of 90 mg/ml, 80 mg/ml, 70 mg/ml, 60 mg/ml, 50 mg/ml, 40 mg/ml, 30 mg/ml, 20 mg/ml, or 10 mg/ml, or a concentration within a range defined by any two of the foregoing values. In a preferred embodiment, the concentration of the matrix is 65 mg/ml. In some embodiments, the concentration of the matrix containing the fibroblasts and keratinocytes or the matrix containing the reshaped and resized skin sample is no greater than 10 mg/ml. For example, in some embodiments, the concentration of the matrix is no greater than one of the values of 10 mg/ml, 9 mg/ml, 8 mg/ml, 7 mg/ml, 6 mg/ml, 5 mg/ml, 4 mg/ml, 3 mg/ml, 2 mg/ml or 1 mg/ml, or a concentration within a range defined by any two of the foregoing values.

In some embodiments, the matrix comprising the fibroblasts and keratinocytes or the matrix comprising the reshaped and resized skin sample is a fluid or gel. In some embodiments, the matrix comprising the fibroblasts and keratinocytes or the matrix comprising the reshaped and resized skin sample is a fluid or gel that desirably conforms to the surface to which the skin graft composition is applied, such as a wound.

In some embodiments, the method further comprises culturing the matrix comprising the fibroblasts and keratinocytes or the matrix comprising the reshaped and resized skin sample. The matrix containing the fibroblasts and keratinocytes or the matrix comprising the reshaped and resized skin sample can be cultured for any suitable amount of time and under any suitable conditions. In some embodiments, the matrix containing the fibroblasts and keratinocytes or the matrix comprising the reshaped and resized skin sample is cultured for up to a length of time of 4 hours, 8 hours, 12 hours, 16 hours, 20 hours, 24 hours, 28 hours, 32 hours, 36 hours, 40 hours, 44 hours, 48 hours, 52 hours, 56 hours, 60 hours, 64 hours, 68 hours, or 72 hours, or for a time within a range defined by any two of the foregoing values. In some embodiments, the matrix containing the fibroblasts and the keratinocytes or the matrix comprising the reshaped and resized skin sample is cultured for 24-28 hours. In some embodiments, the matrix containing the fibroblasts and the keratinocytes or the matrix comprising the reshaped and resized skin sample is cultured at a temperature of 5-40° C., e.g., 5-37° C. In some embodiments, the matrix containing the fibroblasts and the keratinocytes or the matrix comprising the reshaped and resized skin sample is cultured at a temperature of 20-40° C. For example, in some embodiments, the matrix containing the fibroblasts and the keratinocytes or the matrix comprising the reshaped and resized skin sample is cultured at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or at a temperature within a range defined by any two of the foregoing values. In a preferred embodiment, the matrix containing the fibroblasts and the keratinocytes or the matrix comprising the reshaped and resized skin sample is cultured at a temperature of 37° C. In some embodiments, the matrix containing the fibroblasts and keratinocytes or the matrix comprising the reshaped and resized skin sample is cultured in an environment of 0-20% CO₂. In some embodiments, the matrix containing the fibroblasts and keratinocytes or the matrix comprising the reshaped and resized skin sample is cultured in an environment of 0-10% CO₂, e.g., 0% CO₂, 1% CO₂, 2% CO₂, 3% CO₂, 4% CO₂, 5% CO₂, 6% CO₂, 7% CO₂, 8% CO₂, 9% CO₂, or 10% CO₂, or in an environment with a CO₂ concentration within a range defined by any two of the foregoing values. In a preferred embodiment, the matrix containing the fibroblasts and keratinocytes or the matrix comprising the reshaped and resized skin sample is cultured in an environment of 5% CO₂.

In other embodiments, the method does not comprise culturing the matrix comprising the fibroblasts and keratinocytes or the matrix comprising the reshaped and resized skin sample. In some embodiments, the fibroblasts and keratinocytes are modified only as described herein. In other embodiments, the reshaped and resized skin sample is modified only as described herein. For example, in some embodiments, the fibroblasts and keratinocytes are only separated from the skin sample to obtain a solution comprising fibroblasts and keratinocytes, and the solution comprising the fibroblasts and the keratinocytes is combined with a matrix to thereby form a skin graft composition. In other embodiments, the skin sample is only reshaped and resized to obtain a solution comprising the reshaped and resized skin sample, and the solution comprising the reshaped and resized skin sample is combined with a matrix to thereby form a skin graft composition.

The invention provides a skin graft composition. In an embodiment, the skin graft composition is prepared by any of the methods described herein. In another embodiment, the skin graft composition comprises a matrix and a skin sample in which all, or at least substantially all, of the cells of the skin sample are lysed. The lysed cells of the skin sample can be uniformly dispersed in the matrix.

In yet another embodiment, the skin graft composition comprises (a) a matrix and (b) fibroblasts and keratinocytes that have been separated from a skin sample, wherein the composition contains less than 10,000 hair follicles, melanocytes, adipocytes, capillaries, and non-fibroblast connective tissue cells per ml. For example, in some embodiments, the skin graft composition contains less than 10,000, less than 9,000, less than 8,000, less than 7,000, less than 6,000, less than 5,000, less than 4,000, less than 3,000, less than 2,000, or less than 1,000 hair follicles, melanocytes, adipocytes, capillaries, and non-fibroblast connective tissue cells per ml, or an amount of hair follicles, melanocytes, adipocytes, capillaries, and non-fibroblast connective tissue cells per ml within a range defined by any two of the foregoing values.

In yet another embodiment, the skin graft composition comprises (a) a matrix and (b) a reshaped and resized skin sample, wherein the composition contains less than 10,000 hair follicles, melanocytes, adipocytes, capillaries, and non-fibroblast connective tissue cells per ml. For example, in some embodiments, the skin graft composition contains less than 10,000, less than 9,000, less than 8,000, less than 7,000, less than 6,000, less than 5,000, less than 4,000, less than 3,000, less than 2,000, or less than 1,000 hair follicles, melanocytes, adipocytes, capillaries, and non-fibroblast connective tissue cells per ml, or an amount of hair follicles, melanocytes, adipocytes, capillaries, and non-fibroblast connective tissue cells per ml within a range defined by any two of the foregoing values.

In any embodiment, the skin graft composition can be free or substantially free of bacteria or optionally any other microbes. Substantially free of bacteria or optionally any other microbes refers to fewer than 100 bacteria or any other microbes per ml, e.g., fewer than one of the amounts of bacteria or optionally any other microbes of 100, 90, 80, 70, 60, 50, 40, 30, 20, or 10, or a number of bacteria or any other microbes within a range defined by any two of the foregoing values.

In any embodiment, the fibroblasts and the keratinocytes can be uniformly dispersed in the matrix.

The skin graft composition provided by the invention can be for use in the preparation of a skin graft on a subject.

The invention provides a method of providing a skin layer patch or dressing, which can be a skin graft, to a subject comprising providing a skin graft composition in accordance with a method as described herein, and applying the skin graft composition to a surface of the subject that requires a skin layer patch or dressing.

The invention provides a method of preparing a skin layer patch or dressing comprising providing a skin graft composition as described herein, and applying the skin graft composition to any suitable surface. In some embodiments, the surface on a subject is a surface in need of coverage and protection that would typically be provided by healthy skin. For example, the surface on a subject can be a surface that requires a skin layer patch or dressing, such as a surface that requires a skin graft. In some embodiments, the surface is an open wound. In some embodiments, the surface is a chronic wound. In other embodiments, the surface is a surface in need of epithelialization, such as a surface with wrinkles.

In embodiments of the invention, the skin graft composition is applied to the surface of a subject that requires a skin layer patch or dressing, such as a surface of a subject that requires a skin graft, in any suitable manner and by any suitable means. For example, the skin graft composition can be extruded from a press tube onto the surface of the subject. Any suitable press tube can be used. In some embodiments, the press tube is a syringe. In some embodiments, the press tube is any cartridge with a plunger. In some embodiments, the press tube is a pouch. In other embodiments, the skin graft composition is applied by spreading the skin graft composition onto the surface of the subject using any suitable tool such as a spatula. In other embodiments, the skin graft composition is applied by pouring the skin graft composition onto the surface of the subject.

### EMBODIMENTS

1. A method for preparing a skin graft composition comprising: (a) obtaining, from a subject requiring a skin layer patch or dressing, a skin sample, (b) placing the skin sample in a container with a sterile medium, (c) separating fibroblasts and keratinocytes from the skin sample to obtain a solution comprising the fibroblasts and the keratinocytes or reshaping and resizing the skin sample to obtain a solution comprising the reshaped and resized skin sample, and (d) combining the solution comprising the fibroblasts and the keratinocytes or the solution comprising the reshaped and resized skin sample with a matrix to thereby form a skin graft composition.

2. The method of embodiment 1, wherein fibroblasts and keratinocytes from the skin sample are separated to obtain a solution comprising the fibroblasts and the keratinocytes, and the solution comprising the fibroblasts and the keratinocytes is combined with a matrix to thereby form a skin graft composition.

3. The method of embodiment 2, wherein the fibroblasts and keratinocytes are viable after separation of the fibroblasts and keratinocytes from the skin sample.

4. The method of embodiment 2 or 3, wherein the skin sample is dissected prior to placing the skin sample in a container with a sterile medium.

5. The method of any one of embodiments 2-4, wherein the skin sample is at least 1 mm.

6. The method of any one of embodiments 2-5, wherein the container is a glass or plastic container.

7. The method of any one of embodiments 2-5, wherein the container is a cryovial or test tube.

8. The method of any one of embodiments 2-7, wherein the sterile medium is a saline solution.

9. The method of embodiment 8, wherein the saline solution is phosphate buffered saline (PBS).

10. The method of any one of embodiments 2-7, wherein the sterile medium is Dulbecco's Modified Eagle Medium (DMEM)

11. The method of any one of embodiments 2-10, wherein the amount of sterile medium is 1-10000 μl per mm² skin sample.

12. The method of any one of embodiments 2-11, wherein the skin sample is maintained in the container with the sterile medium for up to 72 hours prior to separating the fibroblasts and the keratinocytes from the skin sample.

13. The method of any one of embodiments 2-12, wherein the skin sample is maintained in the container with the sterile medium at a temperature of 5-37° C. prior to separating the fibroblasts and the keratinocytes from the skin sample.

14. The method of any one of embodiments 2-13, wherein the method further comprises adding additional sterile medium to the container comprising the skin sample and the sterile medium prior to separating the fibroblasts and the keratinocytes from the skin sample.

15. The method of embodiment 14, wherein the amount of additional sterile medium is 1-10000 μl per mm² skin sample.

16. The method of any one of embodiments 2-15, wherein the fibroblasts are separated and the keratinocytes are separated from the skin sample by chemical or enzymatic extraction.

17. The method of any one of embodiments 2-15, wherein the fibroblasts are separated and the keratinocytes are separated from the skin sample by mechanical extraction.

18. The method of embodiment 17, wherein the mechanical extraction comprises shaking the container comprising the skin sample and the sterile medium.

19. The method of embodiment 18, wherein the mechanical extraction comprises stirring the skin sample and the sterile medium in the container.

20. The method of embodiment 19, wherein the skin sample and the sterile medium are stirred in the container for up to 1800 seconds.

21. The method of any one of embodiments 2-20, wherein the method further comprises filtering and/or centrifuging the composition containing the fibroblasts and the keratinocytes to provide a purified solution of the fibroblasts and the keratinocytes.

22. The method of embodiment 21, wherein the composition containing the fibroblasts and the keratinocytes is passed through a filter to provide the purified solution of the fibroblasts and the keratinocytes.

23. The method of embodiment 22, wherein the filter has a pore size of 10-100 microns.

24. The method of embodiment 21, wherein the composition containing the fibroblasts and the keratinocytes is centrifuged to provide the purified solution of the fibroblasts and the keratinocytes.

25. The method of embodiment 24, wherein the composition containing the fibroblasts and the keratinocytes is centrifuged at 1-300 g.

26. The method of embodiment 24 or 25, wherein the composition containing the fibroblasts and the keratinocytes is centrifuged for 1-60 minutes.

27. The method of any one of embodiments 24-26, wherein the purified solution of the fibroblasts and the keratinocytes is centrifuged at least one additional time.

28. The method of embodiment 27, wherein the purified solution of the fibroblasts and the keratinocytes is centrifuged at least one additional time at 1-300 g.

29. The method of embodiment 27 or 28, wherein the purified solution of the fibroblasts and the keratinocytes is centrifuged at least one additional time for 1-60 minutes.

30. The method of any one of embodiments 27-29, wherein the method further comprises removing any visible piece of skin from the purified solution of the fibroblasts and the keratinocytes after centrifuging the first time and before centrifuging the second time.

31. The method of embodiment 2, wherein the separation of the fibroblasts and the keratinocytes from the skin sample results in the lysing of all cells of the skin sample.

32. The method of any one of embodiments 2-31, wherein the matrix has a pH of 6-8.

33. The method of embodiment 32, wherein the matrix has a pH of 6.8-7.4.

34. The method of any one of embodiments 2-33, wherein the matrix is a gel matrix.

35. The method of embodiment 34, wherein the gel matrix is a collagen solution.

36. The method of embodiment 35, wherein the collagen solution is a solution of type I, type II, type III, or type IV collagen.

37. The method of embodiment 36, wherein the collagen solution is a solution of type I collagen.

38. The method of any one of embodiments 2-34, wherein the gel matrix is a keratin solution.

39. The method of any one of embodiments 2-34, wherein the gel matrix is a fibrin solution.

40. The method of any one of embodiments 2-34, wherein the gel matrix is a solution comprising collagen, keratin, and fibrin.

41. The method of any one of embodiments 2-40, wherein the concentration of matrix in the matrix containing the fibroblasts and keratinocytes is no greater than 100 mg/ml.

42. The method of any one of embodiments 2-41, wherein the method further comprises culturing the matrix containing the fibroblasts and the keratinocytes.

43. The method of embodiment 42, wherein the matrix containing the fibroblasts and the keratinocytes is cultured for at least 12 hours.

44. The method of embodiment 43, wherein the matrix containing the fibroblasts and the keratinocytes is cultured for 24-48 hours.

45. The method of any one of embodiments 42-44, wherein the matrix containing the fibroblasts and the keratinocytes is cultured at a temperature of 16-40° C.

46. The method of embodiment 45, wherein the matrix containing the fibroblasts and the keratinocytes is cultured at a temperature of 37° C.

47. The method of any one of embodiments 42-46, wherein the matrix containing the fibroblasts and the keratinocytes is cultured in an environment with 0-10% CO₂.

48. The method of embodiment 47, wherein the matrix containing the fibroblasts and the keratinocytes is cultured in an environment with 5% CO₂.

49. A skin graft composition prepared by the method of any one of embodiments 2-48.

50. A skin graft composition comprising a matrix and a skin sample in which all cells of the skin sample are lysed.

51. The skin graft composition of embodiment 50, in which the lysed cells of the skin sample are uniformly dispersed in the matrix.

52. A skin graft composition comprising (a) a matrix and (b) fibroblasts and keratinocytes that have been separated from a skin sample, wherein the composition contains less than 10,000 hair follicles, melanocytes, adipocytes, capillaries, and non-fibroblast connective tissue cells per ml.

53. The skin graft composition of embodiment 52, in which the fibroblasts and keratinocytes are uniformly dispersed in the matrix.

54. A skin graft composition of any one of embodiments 49-53 for use in the preparation of a skin layer patch or dressing on a subject.

55. A method of providing a skin layer patch or dressing to a subject comprising providing a skin graft composition in accordance with the method of any one of embodiments 2-47 and applying the skin graft composition to a surface of the subject that requires a skin layer patch or dressing.

56. The method of embodiment 55, wherein the skin graft composition is extruded from a press tube onto the surface on the subject that requires a skin layer patch or dressing.

57. A method of preparing a skin layer patch or dressing comprising providing a skin graft composition of any one of embodiments 49-53 and applying the skin graft composition to a surface.

58. The method of embodiment 57, wherein a surface on the subject that requires a skin layer patch or dressing.

59. The method of embodiment 58, wherein the skin graft composition is extruded from a press tube onto the surface on the subject that requires a skin graft.

60. The method of embodiment 1, wherein the skin sample is reshaped and resized to obtain a solution comprising the reshaped and resized skin sample and the solution comprising the reshaped and resized skin sample is combined with a matrix to thereby form a skin graft composition.

61. The method of embodiment 60, wherein the skin sample is dissected prior to placing the skin sample in a container with a sterile medium.

62. The method of embodiment 60 or 61, wherein the skin sample is at least 1 mm².

63. The method of any one of embodiments 60-62, wherein the container is a glass or plastic container.

64. The method of any one of embodiments 60-63, wherein the container is a cryovial or test tube.

65. The method of any one of embodiments 60-64, wherein the sterile medium is a saline solution.

66. The method of embodiment 65, wherein the saline solution is phosphate buffered saline (PBS).

67. The method of any one of embodiments 60-66, wherein the sterile medium is Dulbecco's Modified Eagle Medium (DMEM).

68. The method of any one of embodiments 60-67, wherein the amount of sterile medium is 1-10000 μl per mm² skin sample.

69. The method of any one of embodiments 60-68, wherein the skin sample is maintained in the container with the sterile medium for up to 72 hours prior to reshaping and resizing the skin sample.

70. The method of any one of embodiments 60-69, wherein the skin sample is maintained in the container with the sterile medium at a temperature of 5-37° C. prior to reshaping and resizing the skin sample.

71. The method of any one of embodiments 60-70, wherein the method further comprises adding additional sterile medium to the container comprising the skin sample and the sterile medium prior to reshaping and resizing the skin sample.

72. The method of embodiment 71, wherein the amount of additional sterile medium is 1-10000 μl per mm² skin sample.

73. The method of any one of embodiments 60-72, wherein the skin sample is reshaped and resized by dessication of the skin sample.

74. The method of any one of embodiments 60-73, wherein the skin sample is removed from the container and the sterile medium before reshaping and resizing of the skin sample and replaced into the container and the sterile medium after reshaping and resizing of the skin sample.

75. The method of any one of embodiments 60-74, wherein the method further comprises filtering and/or centrifuging the composition containing reshaped and resized skin sample to provide a purified solution of the reshaped and resized skin sample.

76. The method of embodiment 75, wherein the composition containing the reshaped and resized skin sample is passed through a filter to provide the purified solution of the reshaped and resized skin sample.

77. The method of embodiment 76, wherein the filter has a pore size of 10-100 microns.

78. The method of embodiment 76, wherein the composition containing the reshaped and resized skin sample is centrifuged to provide the purified solution of the reshaped and resized skin sample.

79. The method of embodiment 78, wherein the composition containing the reshaped and resized skin sample is centrifuged at 1-300 g.

80. The method of embodiment 78 or 79, wherein the composition containing the reshaped and resized skin sample is centrifuged for 1-60 minutes.

81. The method of any one of embodiments 78-80, wherein the purified solution of the reshaped and resized skin sample is centrifuged at least one additional time.

82. The method of embodiment 81, wherein the purified solution of the reshaped and resized skin sample is centrifuged at least one additional time at 1-300 g.

83. The method of embodiment 81 or 82, wherein the purified solution of the reshaped and resized skin sample is centrifuged at least one additional time for 1-60 minutes.

84. The method of any one of embodiments 60-83, wherein the method further comprises lysing all of the cells of the skin sample.

85. The method of any one of embodiments 60-84, wherein the matrix has a pH of 6-8.

86. The method of embodiment 85, wherein the matrix has a pH of 6.8-7.4.

87. The method of any one of embodiments 60-86, wherein the matrix is a gel matrix.

88. The method of embodiment 87, wherein the gel matrix is a collagen solution.

89. The method of embodiment 88, wherein the collagen solution is a solution of type I, type II, type III, or type IV collagen.

90. The method of embodiment 89, wherein the collagen solution is a solution of type I collagen.

91. The method of any one of embodiments 60-87, wherein the gel matrix is a keratin solution.

92. The method of any one of embodiments 60-87, wherein the gel matrix is a fibrin solution.

93. The method of any one of embodiments 60-87, wherein the gel matrix is a solution comprising collagen, keratin, and fibrin.

94. The method of any one of embodiments 60-93, wherein the concentration of matrix in the matrix containing the reshaped and resized skin sample is no greater than 100 mg/ml.

95. The method of any one of embodiments 60-94, wherein the method further comprises culturing the matrix containing the reshaped and resized skin sample.

96. The method of embodiment 95, wherein the matrix containing the reshaped and resized skin sample is cultured for at least 12 hours.

97. The method of embodiment 96, wherein the matrix containing the reshaped and resized skin sample is cultured for 24-48 hours.

98. The method of any one of embodiments 95-97, wherein the matrix containing the reshaped and resized skin sample is cultured at a temperature of 16-40° C.

99. The method of embodiment 98, wherein the matrix containing the reshaped and resized skin sample is cultured at a temperature of 37° C.

100. The method of any one of embodiments 95-99, wherein the matrix containing the reshaped and resized skin sample is cultured in an environment with 0-10% CO₂.

101. The method of embodiment 100, wherein the matrix containing the reshaped and resized skin sample is cultured in an environment with 5% CO₂.

102. A skin graft composition prepared by the method of any one of embodiments 60-100.

103. A skin graft composition comprising (a) a matrix and (b) a reshaped and resized skin sample, wherein the composition contains less than 10,000 hair follicles, melanocytes, adipocytes, capillaries, and non-fibroblast connective tissue cells per ml.

104. A skin graft composition of embodiment 102 or 103 for use in the preparation of a skin layer patch or dressing on a subject.

105. A method of providing a skin layer patch or dressing to a subject comprising providing a skin graft composition in accordance with the method of any one of embodiments 60-100 and applying the skin graft composition to a surface of the subject that requires a skin layer patch or dressing.

106. The method of embodiment 105, wherein the skin graft composition is extruded from a press tube onto the surface on the subject that requires a skin layer patch or dressing.

107. A method of preparing a skin layer patch or dressing comprising providing a skin graft composition of embodiment 103 or 104 and applying the skin graft composition to a surface.

108. The method of embodiment 107, wherein a surface on the subject that requires a skin layer patch or dressing.

109. The method of embodiment 108, wherein the skin graft composition is extruded from a press tube onto the surface on the subject that requires a skin graft.

### Example 1

This Example demonstrates the preparation of an exemplary skin graft composition of the present invention.

5.0 ml of collagen was placed in a sterile tube. 0.37 ml of titration buffer was added to the sterile tube in the center of the collagen using a pipette. The tube was inverted until thoroughly mixed (about five times). After 30 minutes, the pH was recorded to ensure the collagen is neutralized (i.e., at a pH as described herein).

A 1 cm² retro auricular (behind the ear) skin sample was obtained from a patient. To obtain the skin sample, lidocaine was administered to the area to anesthetize it, and then a scalpel was used to obtain the skin sample. The skin sample was stored at room temperature in a vial with sterile PBS.

The skin sample was then processed to create the skin graft composition. The skin sample was removed from the vial, and the PBS was discarded. The skin sample was washed with PBS 10 times by pipetting. Using a scalpel, the skin sample was dissected to obtain the required number of tissue pieces. Four pieces of skin sample, which were each approximately 5 mm in diameter, were obtained. If the skin sample contained a subcutaneous fat layer, the scalpel was used to carefully remove/scape off the fat.

The four pieces of skin tissue were placed in a C-tube, and 1 ml of PBS was added. The C-tube was placed in a GENTLEMACS™ dissociator, and the pre-programmed m_spleen_01 program on the GENTLEMACS™ dissociator was run. The m_spleen_-01 program runs for 56 seconds, in which the rotator switches direction from clockwise to counterclockwise and back again every 1-2 s. The C-tube was then centrifuged for 10 minutes at 1200 rpm. Using a pipette, 750 μl of supernatant was removed from the C-tube and discarded. The remaining supernatant, which contained the pellet, in the C-tube was no more than 300 μl. Using a pipette, the pellet, which contained fibroblasts and keratinocytes, was transferred to a tube containing the neutralized collagen to create the skin graft composition. The skin graft composition was then stored at ambient conditions (at room temperature, i.e., between 15-25° C., at a humidity between 30-60% avoiding direct exposure to light).

### Example 2

This Example demonstrates the efficacy of the inventive skin graft compositions.

Skin graft compositions were prepared as described in Example 1. A physician applied the skin graft composition on a patient's wound, which was then covered with a porous silicon membrane and/or gauze. The silicone membrane and/or gauze were changed as needed. Four patients were treated with the inventive skin graft compositions as follows:

Patient 1 was 43 years old and had tissue necrosis after an aesthetic surgical procedure in her lower abdomen and belly button. Patient 2 was 51 years old and had an ulcer caused by a wound that never healed from an accident five years ago on the ankle and part of the left foot. Patient 3 was 26 years old and had healing wounds following a surgical procedure in the breasts. Patient 3 took anticoagulant medication. Patient 4 had wounds that did not close due to a surgical procedure in the breasts. Measurements were taken of the wounds prior to treatment and a certain number of days after treatment. Patients 1 and 2's wounds were measured 30 days after treatment. Patient 3's wounds were measured 34 days after treatment. Patient 4's wounds were measured 15 days after treatment. Table 1 reports the wound measurements. Epithelialized indicates that the wounds were closed.

TABLE 1

| Patient | Wound | Area at Day 1 (cm²) | Area Post-Treatment (cm²) |
|---|---|---|---|
| 1 | 1 | 7.12 + 2 cm deep | 1.22 + 0 cm deep |
| | 2 | 2.72 | Epithelialized |
| | 3 | 0.88 | Epithelialized |
| 2 | 1 | 33.09 | 19.9 |
| 3 | 1 | 2.21 | Epithelialized |
| | 2 | 2.13 | Epithelialized |
| 4 | 1 | 5.44 | 5.4 |
| | 2 | 4.30 | 3.79 |

Patient 2 also reported a 50% decrease in pain and inflammation. The wound at day 30 was 60% closed. In patient 4, wound 2 decreased by 12% in area (equivalent to 0.51 cm²). Wound 1 decreased by 1% in area. Wounds 1 and 2 both decreased in depth.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for preparing a skin graft composition comprising:
(a) obtaining, from a subject requiring a skin layer patch or dressing, a skin sample,
(b) placing the skin sample in a container with a sterile medium,
(c) separating fibroblasts and keratinocytes from the skin sample to obtain a solution comprising the fibroblasts and the keratinocytes or reshaping and resizing the skin sample to obtain a solution comprising the reshaped and resized skin sample, and
(d) combining the solution comprising the fibroblasts and the keratinocytes or the solution comprising the reshaped and resized skin sample with a matrix to thereby form a skin graft composition.

2. The method of claim 1, wherein fibroblasts and keratinocytes from the skin sample are separated to obtain a solution comprising the fibroblasts and the keratinocytes, and the solution comprising the fibroblasts and the keratinocytes is combined with a matrix to thereby form a skin graft composition.

3. The method of claim 2, wherein the fibroblasts and keratinocytes are viable after separation of the fibroblasts and keratinocytes from the skin sample.

4. The method of claim 2, wherein the skin sample is dissected prior to placing the skin sample in a container with a sterile medium.

5. The method of claim 2, wherein the skin sample is at least 1 mm².

6. The method of claim 2, wherein the container is a glass or plastic container.

7. The method of claim 2, wherein the container is a cryovial or test tube.

8. The method of claim 2, wherein the sterile medium is a saline solution or Dulbecco's Modified Eagle Medium (DMEM).

9. The method of claim 8, wherein the saline solution is phosphate buffered saline (PBS).

10. (canceled)

11. The method of claim 2, wherein the amount of sterile medium is 1-10000 μl per mm² skin sample.

12. The method of claim 2, wherein the skin sample is maintained in the container with the sterile medium for up to 72 hours prior to separating the fibroblasts and the keratinocytes from the skin sample.

13. The method of claim 2, wherein the skin sample is maintained in the container with the sterile medium at a temperature of 5-37° C. prior to separating the fibroblasts and the keratinocytes from the skin sample.

14. The method of claim 2, wherein the method further comprises adding additional sterile medium to the container comprising the skin sample and the sterile medium prior to separating the fibroblasts and the keratinocytes from the skin sample.

15. The method of claim 14, wherein the amount of additional sterile medium is 2-10000 μl per mm² skin sample.

16. The method of claim 2, wherein the fibroblasts are separated and the keratinocytes are separated from the skin sample by chemical, enzymatic, or mechanical extraction.

17. (canceled)

18. The method of claim 16, wherein the mechanical extraction comprises shaking or stirring the container comprising the skin sample and the sterile medium.

19. (canceled)

20. The method of claim 18, wherein the skin sample and the sterile medium are stirred in the container for up to 1800 seconds.

21. The method of claim 2, wherein the method further comprises filtering and/or centrifuging the composition containing the fibroblasts and the keratinocytes to provide a purified solution of the fibroblasts and the keratinocytes.

22. The method of claim 21, wherein the composition containing the fibroblasts and the keratinocytes is passed through a filter to provide the purified solution of the fibroblasts and the keratinocytes.

23. The method of claim 22, wherein the filter has a pore size of 10-100 microns.

24. The method of claim 21, wherein the composition containing the fibroblasts and the keratinocytes is centrifuged to provide the purified solution of the fibroblasts and the keratinocytes.

25. The method of claim 24, wherein the composition containing the fibroblasts and the keratinocytes is centrifuged at 1-300 g.

26. The method of claim 24, wherein the composition containing the fibroblasts and the keratinocytes is centrifuged for 1-60 minutes.

27. The method of claim 24, wherein the purified solution of the fibroblasts and the keratinocytes is centrifuged at least one additional time.

28. The method of claim 27, wherein the purified solution of the fibroblasts and the keratinocytes is centrifuged at least one additional time at 1-300 g.

29. The method of claim 27, wherein the purified solution of the fibroblasts and the keratinocytes is centrifuged at least one additional time for 1-60 minutes.

30. The method of claim 27, wherein the method further comprises removing any visible piece of skin from the purified solution of the fibroblasts and the keratinocytes after centrifuging the first time and before centrifuging the second time.

31. The method of claim 2, wherein the separation of the fibroblasts and the keratinocytes from the skin sample results in the lysing of all cells of the skin sample.

32. The method of claim 2, wherein the matrix has a pH of 6-8.

33. The method of claim 32, wherein the matrix has a pH of 6.8-7.4.

34. The method of claim 2, wherein the matrix is a gel matrix.

35. The method of claim 34, wherein the gel matrix is a collagen, keratin, and/or fibrin solution.

36. The method of claim 35, wherein the collagen solution is a solution of type I, type II, type III, or type IV collagen.

37. (canceled)

38. (canceled)

39. (canceled)

40. (canceled)

41. The method of claim 2, wherein the concentration of matrix in the matrix containing the fibroblasts and keratinocytes is no greater than 100 mg/ml.

42. The method of claim 2, wherein the method further comprises culturing the matrix containing the fibroblasts and the keratinocytes.

43. The method of claim 42, wherein the matrix containing the fibroblasts and the keratinocytes is cultured for at least 12 hours.

44. The method of claim 43, wherein the matrix containing the fibroblasts and the keratinocytes is cultured for 24-48 hours.

45. The method of claim 42, wherein the matrix containing the fibroblasts and the keratinocytes is cultured at a temperature of 16-40° C.

46. The method of claim 45, wherein the matrix containing the fibroblasts and the keratinocytes is cultured at a temperature of 37° C.

47. The method of claim 42, wherein the matrix containing the fibroblasts and the keratinocytes is cultured in an environment with 0-10% CO₂.

48. The method of claim 47, wherein the matrix containing the fibroblasts and the keratinocytes is cultured in an environment with 5% CO₂.

49. A skin graft composition prepared by the method of claim 2.

50. A skin graft composition comprising a matrix and a skin sample in which all cells of the skin sample are lysed.

51. The skin graft composition of claim 50, in which the lysed cells of the skin sample are uniformly dispersed in the matrix.

52. A skin graft composition comprising (a) a matrix and (b) fibroblasts and keratinocytes that have been separated from a skin sample, wherein the composition contains less than 10,000 hair follicles, melanocytes, adipocytes, capillaries, and non-fibroblast connective tissue cells per ml.

53. The skin graft composition of claim 52, in which the fibroblasts and keratinocytes are uniformly dispersed in the matrix.

54. (canceled)

55. A method of providing a skin layer patch or dressing to a subject comprising providing a skin graft composition in accordance with the method of claim 2 and applying the skin graft composition to a surface of the subject that requires a skin layer patch or dressing.

56. The method of claim 55, wherein the skin graft composition is extruded from a press tube onto the surface on the subject that requires a skin layer patch or dressing.

57. A method of preparing a skin layer patch or dressing comprising providing a skin graft composition of claim 49 and applying the skin graft composition to a surface.

58. (canceled)

59. (canceled)

60. The method of claim 1, wherein the skin sample is reshaped and resized to obtain a solution comprising the reshaped and resized skin sample and the solution comprising the reshaped and resized skin sample is combined with a matrix to thereby form a skin graft composition.

61.-102. (canceled)

103. A skin graft composition comprising (a) a matrix and (b) a reshaped and resized skin sample, wherein the composition contains less than 10,000 hair follicles, melanocytes, adipocytes, capillaries, and non-fibroblast connective tissue cells per ml.

104. (canceled)

105. A method of providing a skin layer patch or dressing to a subject comprising providing a skin graft composition in accordance with the method of claim 60 and applying the skin graft composition to a surface of the subject that requires a skin layer patch or dressing.

106. (canceled)

107. A method of preparing a skin layer patch or dressing comprising providing a skin graft composition of claim 103 and applying the skin graft composition to a surface.

108. (canceled)

109. (canceled)
